(19) United States
(12) Patent Application Publication
(10) Pub. No.: US 2017/0203044 A1
(43) Pub. Date: Jul. 20, 2017

(54) PLUNGER ROD FOR MEDICAL PACKAGING

(71) Applicant: Schott Schweiz AG, St. Gallen (CH)

(72) Inventor: Christian Komann, St. Gallen (CH)

(73) Assignee: Schott Schweiz AG, St. Gallen (CH)

(21) Appl. No.: 15/405,817

(22) Filed: Jan. 13, 2017

(30) Foreign Application Priority Data
Jan. 15, 2016 (DE) 10 2016 200 434.4
Jan. 2, 2017 (EP) 17150016.8

(51) Int. Cl. A61M 5/315; A61M 5/28; A61M 5/31

(52) U.S. Cl. CPC A61M 5/31511; A61M 5/3129; A61M 5/28

(57) ABSTRACT

A plunger rod for medical packaging such as syringe barrels or cartridges. The plunger rod includes a first and a second plunger rod end, a torsional axis, and at least one component. Each component includes at least one cross sectional surface which is located at a distance from the torsional axis and increases a bending- and torsional rigidity of the plunger rod.

### BACKGROUND OF THE INVENTION

1. Field of the Invention

The invention relates to medical packaging, and, more particularly to a plunger rod for medical packaging, for example syringe barrels or cartridges, wherein the plunger rod has a first and a second plunger rod end.

2. Description of the Related Art

On syringes, as known for example from U.S. Pat. No. 7,824,380 B2, the plunger rods consist of crossed ribs. In U.S. Pat. No. 7,824,389 B2 the plunger rod end on which the stopper is fastened is additionally equipped with support ribs, so that in case of canting of the plunger rod the plunger rod can be released more easily. Canting of the plunger rod can already occur with minimal insertion of the plunger rod into the syringe barrel. The plunger rods known from the state of the art are essentially a long structural component with high rigidity relative to operating forces. It is however disadvantageous that—in order to keep tooling complexity low—the plunger rod from the state of the art only offers low rigidity relative to torsional stresses. An additional disadvantage can be found in that the distance between the supporting ribs is very large for prevention of critical canting.

U.S. Pat. No. 2,392,104 B discloses a syringe with a glass plunger rod. The glass plunger rod according to U.S. Pat. No. 2,392,104 B can be hollow. A disadvantage of the hollow glass plunger rod from U.S. Pat. No. 2,392,104 B is its high weight. Moreover it is not apparent from U.S. Pat. No. 2,392,104 B how a plunger rod must be designed in order to provide sufficient rigidity relative to torsional stresses.

A syringe with devices for protection against needle strikes and medical substance abuse has become known from DE 38 74 162 T2. The plunger rod in DE 38 74 162 T2 is equipped with safety devices on the outside, to prevent removal of the rod from the distal end of the syringe barrel.

DE 199 23 131 A1 discloses a medical syringe. The syringe according to DE 199 23 131 A1 is in the embodiment of a plastic syringe and includes a plunger rod with a special cross sectional shape to provide the plunger rod with the necessary rigidity. The syringe described in DE 199 23 131 A1 distinguishes itself through high strength due to the rigidity. In contrast hereto however, high bending- and torsional rigidity requires high rigidity relative to torsional loads, and not just high strength.

From U.S. Pat. No. 5,032,114 A, a syringe with a plunger has become known, wherein the plunger comprises a helix. However, the purpose of this helical structure is not described anywhere in U.S. Pat. No. 5,032,114 A. The helical structure, as in U.S. Pat. No. 5,032,114 A is however not rigid relative to torsion, because of which no structure is shown in U.S. Pat. No. 5,032,114 A wherein the torsion resistance is increased.

DE 1 175 146 shows a vessel closure with a resilient spacer that has a helical structure and no syringe.

What is needed in the art is a plunger rod for medical packaging that overcomes the disadvantages of the current state of the art.

### SUMMARY OF THE INVENTION

The present invention provides a plunger rod, for example a hollow plunger rod for medical packaging such as syringe barrels or cartridges wherein the plunger rod includes a torsional axis and cross sectional surfaces, as well as means to increase the bending- and torsional rigidity. The means to increase the bending- and torsional rigidity are components of the plunger rod that include or provide at least one cross sectional surface, at a distance to the torsional axis.

The plunger rod may be produced with the assistance of an injection molding method.

If the plunger rod is produced in an injection molding process, the material of the plunger rod can be a plastic material, for example thermoplastic materials. One suitable product is polyethylene (PE). Another polymer can be polypropylene (PP) or polystyrene (PS) or polyoxymethylene (POM). The materials of the plunger rod can be thermoplastics and differ from the materials of the syringe barrel. In contrast to the plunger rod, the materials of the syringe barrel can be in contact over an extended period of time with a pharmaceutical product contained therein. To be able to store pharmaceutical products over an extended time in the syringe barrel it is necessary to use syringe barrels, consisting largely of inert materials from which no substances can diffuse into the pharmaceutical product that is stored in the syringe barrel. Suitable for this are high quality plastics, such as COC and COP which are hard and relatively brittle. To be able to use the syringe barrels for storage of pharmaceutical products, the materials can be sterilized at temperatures of >100° C., for example 121° C., for example at a maximum 180° C. This would be applicable for cycloolefin polymers (COP) and cycloolefin copolymers (COC). Glass would also be a possible material for the syringe barrel, in place of COC and COP. An additional possible material for the syringe barrel would also be polypropylene (PP) or polyethylene (PE). Although polypropylene (PP) or polyethylene (PE) are softer materials that can absorb stresses, but based on their diffusion characteristics they may be suitable only for short-term storage of pharmaceutical products.

If the plunger rod is designed to be hollow, the plunger rod produced in an injection molding process may be produced with the assistance of an injection mold with a long core that is inserted for example into the plunger rod from the plug side of the plunger rod. A resulting plunger rod is then closed on only one side. Alternatively, a closed hollow barrel could also be produced from a plastic material with the assistance of a gas-assisted injection molding process. The gas-assisted injection molding process is a special injection molding process.

By using a plastic material for the plunger rod instead of a glass material, as disclosed in U.S. Pat. No. 2,392,104 B, considerable reductions in weight can be achieved, especially if also the syringe body designed as a plastic component.

To prevent a plunger rod consisting of a thermoplastic from coming into contact with the pharmaceutical product that is stored in the syringe barrel, a plug may be provided on the plunger rod. The plug may for example consist of rubber. The plug normally has an inside thread and the plunger rod at its distal end has an outside thread, so that the plunger rod can be screwed into the plug.

A first embodiment of the invention provides that the plunger rod includes reinforcement elements, at least in sections, arranged diagonally opposite the torsional axis, for example in the embodiment of reinforcement ribs as a means to increase the bending-and torsional rigidity. The reinforcement elements, for example the reinforcement ribs may be tilted relative to the torsional axis at an angle α, for example >10°, for example 20°, 30°, or 45°. With reinforcement elements that are tilted 45°, the torsional rigidity can be increased by a factor of 5, compared with a conventional plunger rod of same weight, bending rigidity and tooling complexity. With such 45° reinforcement the distances of the support ribs can be reduced at the plunger plug end. Also, since this is a clamping point, the material thickness of the wall can be reduced in the direction of the second plunger rod end with the thrust plate. The plunger rod can have a cross-shaped cross section. High torsional rigidity can be achieved, if the plunger rod has at least one tubular segment in addition to the reinforcement elements, especially the reinforcement ribs.

This tubular segment can not only be provided in addition to the reinforcement ribs, but also alone, to provide the means to increase the bending and torsional rigidity.

The tubular segment can be in the embodiment of a singular segment, extending over the entire plunger rod, or over a substantial part of the plunger rod, for example over 30 to 90% of the plunger rod. Alternatively it is also possible that the plunger rod consists of not only a single tubular segment, but instead of several tubular segments, or of one or several partial sections of a tubular segment. The partial sections are for example 180° segments of a tubular segment, in other words half-tube segments. 90° segments of a tubular segment, in other words quarter-tube segments are also possible. In a first embodiment, the tubular segment has a tube diameter, wherein the tube diameter can be approximately consistent with the outside diameter of the plunger rod, or can be 10 to 90% of the outside diameter of the plunger rod. In addition to reinforcement by the tubular segments it is also possible to provide reinforcement in that additional reinforcement elements are arranged outside and/or inside of the at least one tubular segment.

To prevent canting when inserting the plunger rod into the syringe barrel, and a possibly undesired escape of medication from a filled syringe barrel or cartridge, support elements may be provided in the region of the first plunger rod end with the plunger plug. The plunger is operated with the plunger rod by a thrust plate, located at the second plunger rod end.

With the plunger rod according to the present invention, the torsional rigidity can be increased by various methods. According to the invention, the torsional rigidity can be increased if one or several cross sectional surfaces are provided at a distance from the torsional axis. The torsional rigidity can be further increased, if cross sectional surfaces are as far away as possible from the torsional axis and are provided in great numbers. An additional increase is possible, if the cross sectional surface is distributed continuously over the vertical length of the torsional axis. If the continuously progressing cross sectional surfaces approach the form of several divergently progressing spirals, the torsional rigidity is further increased.

The plunger rod according to the invention can be used in a syringe barrel with a syringe cone or in a cartridge, whereby according to the invention a plunger rod is guided in the syringe barrel or in the cartridge.

### BRIEF DESCRIPTION OF THE DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIGS. 1a-1e illustrate a first embodiment of a plunger rod with a tubular segment and reinforcement elements;

FIGS. 2a-2b illustrate a comparison of an embodiment according to the invention, per FIGS. 1a-1e with a conventional embodiment (FIG. 2b);

FIGS. 3a-3e illustrate an arrangement with a rod, with a tubular element extending over 90% of the plunger rod;

FIGS. 4a-4b illustrate a plunger rod according to FIGS. 3a-3e, in comparison to a conventional plunger rod according to the state of the art (FIG. 4b);

FIGS. 5a-5e illustrate a plunger rod with sectional tubular elements along the plunger rod, as well as additional reinforcement elements;

FIGS. 6a-6e illustrate a plunger rod with sectional tubular elements, as well as a second type of reinforcement elements in the form of honeycomb bodies.

FIGS. 7a-7b illustrate a comparison of a plunger rod according to FIGS. 5a-5b with a plunger rod according to the state of the art (FIG. 7b);

FIGS. 8a-8b illustrate a comparison of a plunger rod according to FIGS. 6a-6b with a plunger rod according to the state of the art (FIG. 8b);

FIGS. 9a-9e illustrate one embodiment of a plunger rod, including reinforcement elements arranged only in sections diagonally opposite the torsional axis; and

FIGS. 10a-10b illustrate a comparison of a plunger rod according to FIGS. 9a-9b with a plunger rod according to the state of the art (FIG. 9b).

Corresponding reference characters indicate corresponding parts throughout the several views. The exemplifications set out herein illustrates embodiments of the invention and such exemplifications are not to be construed as limiting the scope of the invention in any manner.

### DETAILED DESCRIPTION OF THE INVENTION

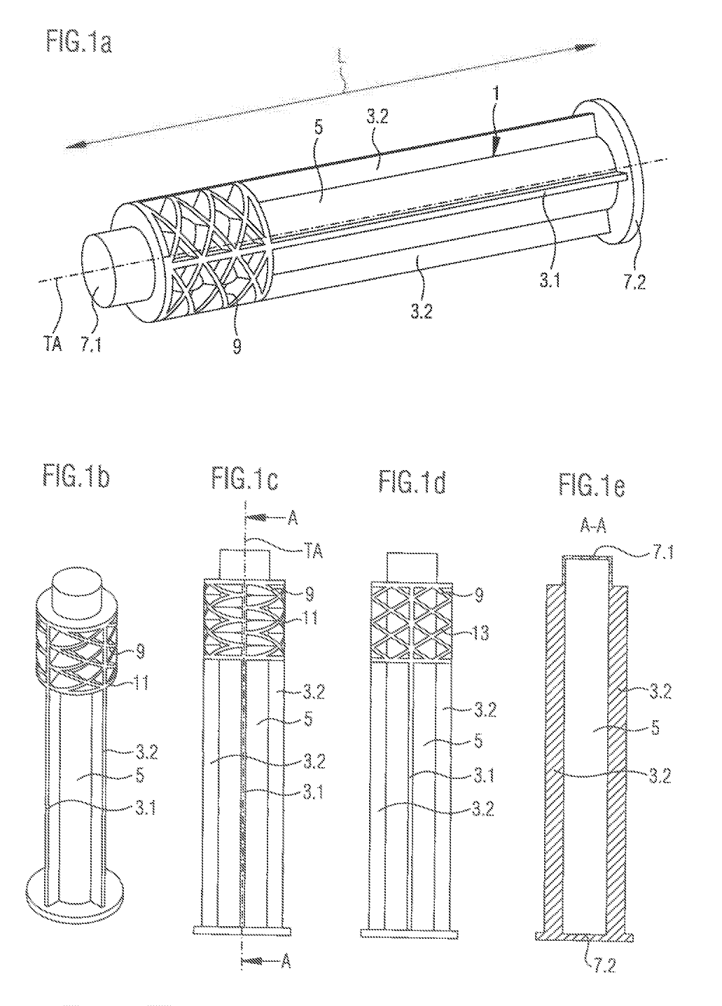

A first embodiment of a plunger rod 1 according to the invention is illustrated in FIGS. 1a-1e, wherein the plunger in its cross section, as in the state of the art includes two crossed ribs 3.1, 3.2. According to the embodiment in FIGS. 1a-1e, the plunger rod also includes a tubular segment 5. In the embodiment according to FIGS. 1a-1e, the tubular segment extends practically over 100% of length L of the plunger rod, from first plunger rod end 7.1 to second plunger rod end 7.2.

Tubular segment 5 provides a cross sectional surface, distant from torsional axis TA, thus increasing the torsion and bending rigidity of the plunger rod. In the embodiment according to FIGS. 1a-1b reinforcement elements 9 are provided in addition to tubular element 5 on the outside of tubular element 5 in the region of first plunger rod end 7.1 that, on the one hand serve to provide additional cross sectional surfaces outside the torsional axis; as well as support ribs in the region of first plunger rod end 7.1 that corresponds with the plunger plug, that ensure that the syringe after insertion into the syringe barrel cannot cant. FIGS. 1b and 1c illustrate a syringe barrel according to FIG. 1a in detail and in various views, whereby same components have been identified with same reference numbers. In the case of an injection molding tool the direction of demolding is consistent with the direction of stamping. The direction of stamping points from the mold core side toward the side of the mold nozzle.

FIG. 1d illustrates the crossed arrangement of reinforcement elements 9. FIG. 1e is a sectional view through the arrangement per FIG. 1c, along line A-A and clearly shows tubular segment 5 in a cross-sectional view. FIG. 1e also shows the plunger rod end 7.1, synonymous with the plug and second plunger rod end 7.2 that can be equated with a pressure plate in the form of a thrust plate that serves to move the plunger rod in the axial direction. Moreover, inferred in FIG. 1e are ribs 3.2 and the fact that the tubular segment 5 is a hollow barrel. Such a completely enclosed hollow barrel can only be produced in a special process, for example an internal gas pressure process. Alternatively, a hollow barrel can also be obtained with the assistance of an injection molding tool with a long core that is inserted into the plunger rod from the plunger plug side. The resulting tube is then closed off on only one side.

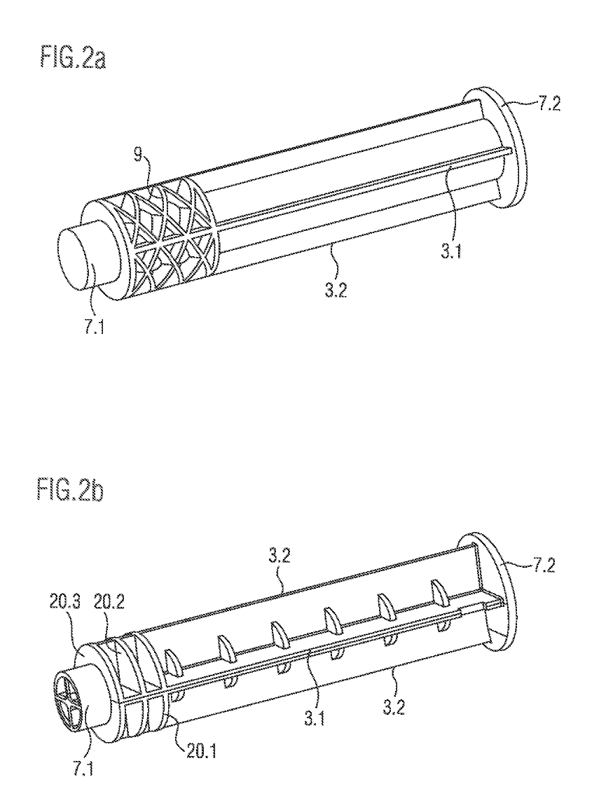

FIGS. 2a and 2b illustrate a comparison of the inventive plunger rod according to FIG 1a. The plunger rod according to FIG. 2a is consistent with the plunger rod according to FIG. 1a. Same components as in FIG. 1a are identified with the same reference numbers. FIG. 2b illustrates a plunger rod according to the state of the art with crossed ribs 3.1, 3.2. The first plunger rod end with the plunger plug is identified with 7.1; the second plunger rod end with the thrust plate is identified with 7.2.

To avoid canting of the plunger rod, as in the state of the art, disks 20.1, 20.2, 20.3 can be provided in the state of the art in the region of the first piston rod end as shown for example in U.S. Pat. No. 7,824,380 B2.

The weight of the plunger rod according to the state of the art is 17 g; the weight of the plunger rod per FIGS. 2a and 1a is 14.5 g. The inventive plunger rod according to FIGS. 2a and 1ahas a torsional stress that is lower by a factor of 11 than the embodiment according to the state of the art (FIG. 2b), and a torsional displacement that is lower by a factor of 16 compared to the embodiment according to the state of the art (FIG. 2b).

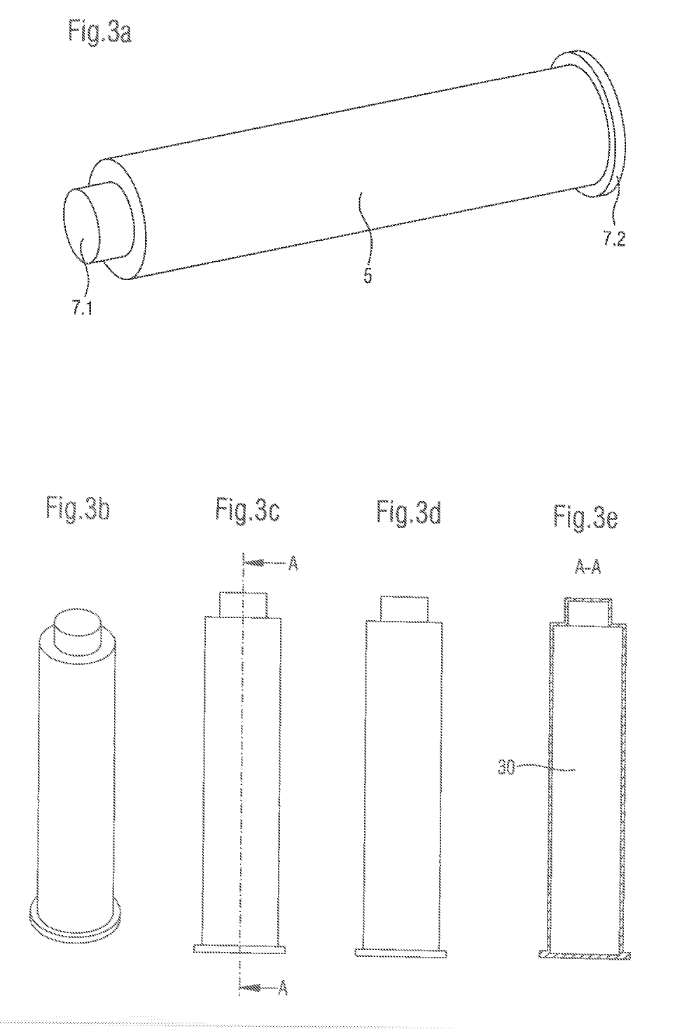

In FIGS. 3a-3e an alternative design of a plunger rod with tubular barrel is depicted. Tubular barrel 5 has a diameter that is substantially consistent with the diameter of the plunger rod. The first plunger rod end, including the plunger plug is identified with 7.1, the second plunger rod end is identified with 7.2.

In the design per FIG. 3a no additional reinforcement elements as in FIGS. 1a-1e are provided. Tilting during insertion of the syringe plunger is avoided in that the tubular barrel has substantially the same outside diameter as the syringe barrel itself. FIGS. 3b-3e are alternative views, including sectional A-A according to FIG. 3e. Same components are identified with the same reference numbers as in FIG. 3a. As can be clearly seen in FIG. 3e, the tubular barrel 5 is hollow on the inside, in other words it has a hollow space 30. The design per FIGS. 3a-3e illustrates another embodiment of a plunger rod. However the embodiment according to FIGS. 3a-3e may be expensive to manufacture, for example due to the high manufacturing costs for the hollow barrel. One possible manufacturing method is the 3D-pressure. The design of the plunger rod according to FIGS. 1a-1e and FIG. 2a can be easier to produce than a complete hollow barrel, since the ribbing provided in the embodiments per FIGS. 1a-1e require minimal tools.

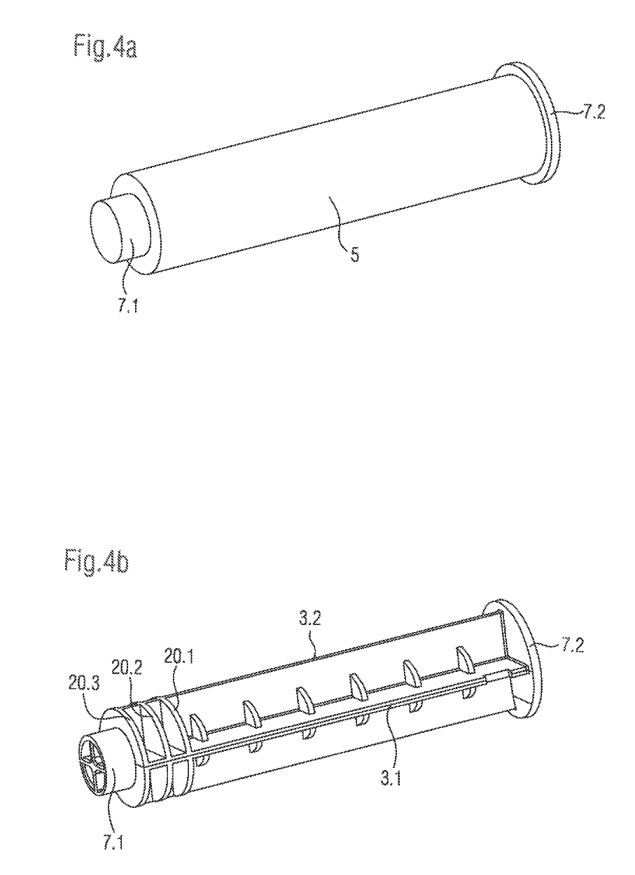

FIGS. 4a and 4b again illustrate the inventive plunger rod according to FIG. 3a, compared to a plunger rod according to the state of the art (FIG. 4b). The plunger rod according to the state of the art (FIG. 4b) is consistent with that in FIG. 2b. In this respect, the reference numbers from FIG. 2b were adopted for FIG. 4b. The weight of the plunger rod according to FIG. 4b is 17 g; the weight of the inventive plunger rod according to FIG. 4a is only 11.46 g due to the fact that the tubular barrel is in the embodiment of a hollow barrel. Compared to the conventional component according to FIG. 4b, the inventive plunger rod per FIG. 4a has a torsional stress that is greater by a factor of 11 than the component according to the state of the art; and a torsional displacement that is greater by a factor of 55.

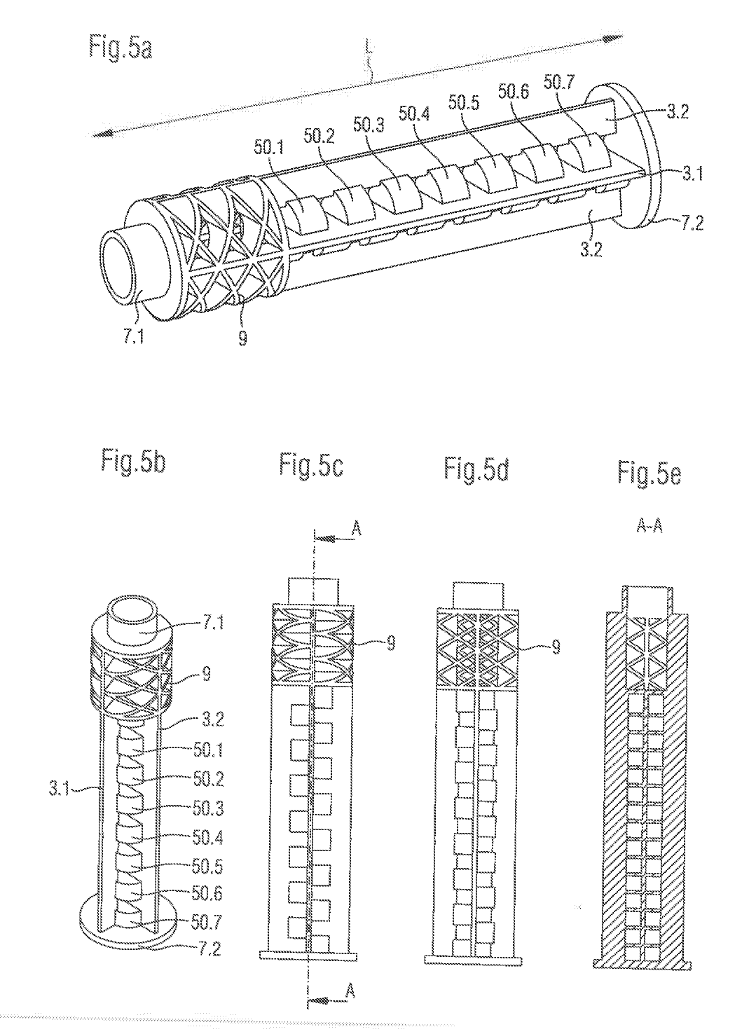

Another embodiment of a plunger rod according to the invention is illustrated in FIGS. 5a-5e. The plunger rod does not include one single tubular segment 5 along its length L, but a multitude of tubular segments 50.1, 50.2, 50.3, 50.4, 50.5, 50.6, 50.7, wherein the present segments are partial segments, that is 90° segments of a tubular segment. The tubular 90° segments in the illustrated embodiments are thus quarter-tubes. Half-tubes, that is 180° segments would also be possible, however 90° segments are generally more effective. This is due to the fact, that an ideal structure of a tube designed for torsional stress with an open tube shell generally includes more contradirectional spirals. Due to this realization, a plunger rod with 90° segments has a more ideal, more uniform flow of forces and greater rigidity than 180° segments. In the design according to FIGS. 5a-5e it is possible with a plunger rod that is produced in a standard injection molding process to increase the torsional rigidity. As is the case in the embodiment per FIG. 1a, additional reinforcement elements 9 are provided in the region of first plunger rod end 7.1. As is the case in FIG. 1a, these are designed as cross-over. Second piston rod end 7.2 is again designed as a thrust plate. Moreover, an opening on the face on plunger rod 7.1 is shown, as well as a hollow space under the reinforcement ribs in that location. In this embodiment, FIGS. 5a-5e illustrate a plunger rod with sectional tubular segments 50.1, 50.2, 50.3, 50.4, 50.5, 50.6, and 50.7 in the form of quarter-tubes. The additional reinforcement elements are identified with 9 and are arranged in the region of the first plunger rod end. FIG. 5d is a section according to intersection A-A in FIG. 5c The individual tubular partial segments 50.1, 50.2, 50.3, 50.4, 50.5, 50.6, as well as the crossed reinforcement elements are easily recognizable.

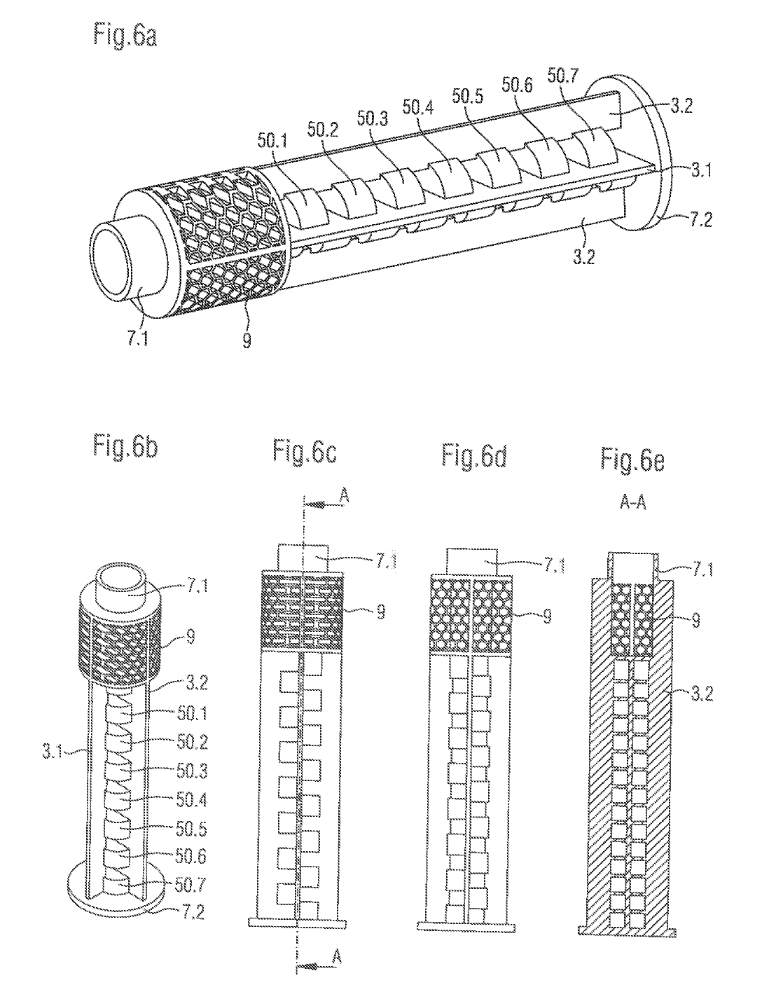

FIGS. 6a-6e show an alternative embodiment to FIGS. 5a-5e, also with tubular partial segments. The partial segments are again 90° segments, in other words quarter-tubes. In contrast to FIGS. 5a-5e, the additional reinforcement elements are designed differently. The additional reinforcement elements 9 in the embodiment according to FIGS. 6a-6e have a honeycomb shape, the tubular partial segments 50.1, 50.2, 50.3, 50.4, 50.5, 50.6, 50.7 are arranged offset to one another as in FIGS. 6a-6e. In principle, the tubular segments 50.1, 50.2, 50.3, 50.4, 50.5, 50.6, 50.7 themselves also represent reinforcement elements. The additional reinforcement elements in the form of honeycombs can reduce the possible tilt angle of the plunger rod in the syringe barrel since the syringe barrel edge makes contact sooner with ribbing during tilting, and since the areas in which there is no ribbing have become much smaller. This positive effect exists also in diagonal ribbing (i.e. 45°). An equivalent reduction of tilting cannot be achieved with simple plates.

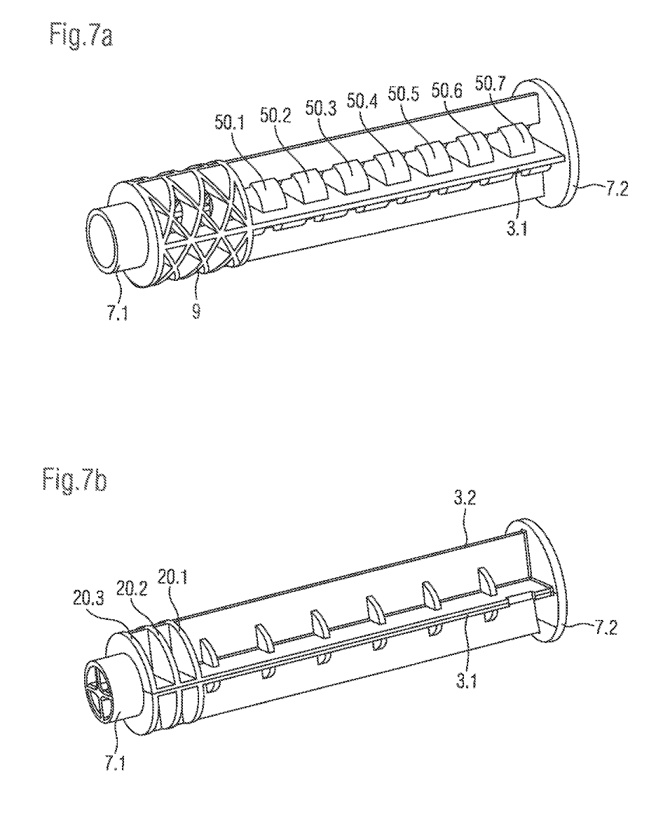

FIGS. 7a and 7b illustrate a comparison of an inventive plunger rod with tubular segments 50.1, 50.2, 50.3, 50.4, 50.5, 50.6, 50.7 according to FIG. 5a, compared to a conventional plunger rod according to FIG. 7b. The weight of the inventive plunger rod is 15.71 g (FIG. 7a); the weight of the conventional plunger rod is 17 g (FIG. 7b). The torsional stress of the inventive plunger rod is lower by a factor of 3 than that of the reference plunger rod according to FIG. 7b. The torsional displacement is lower by a factor of 7.

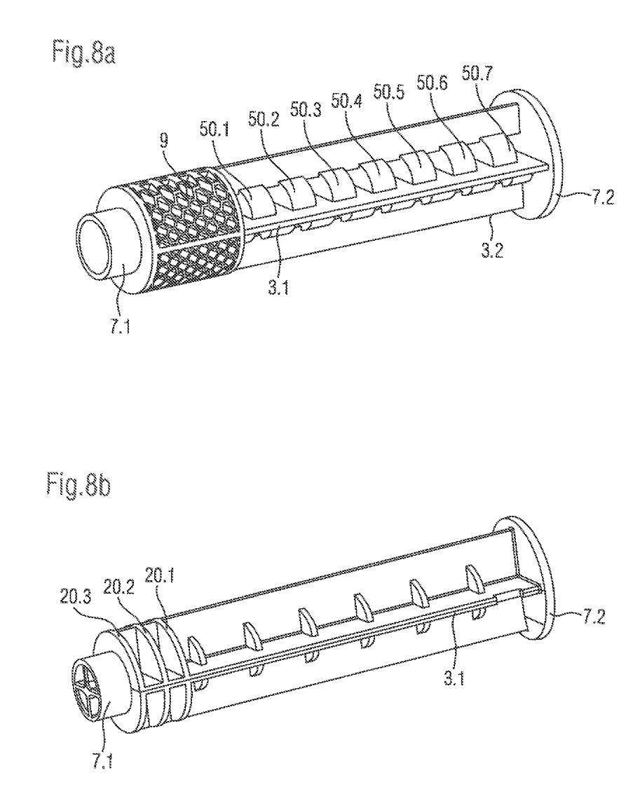

In FIGS. 8a and 8b an inventive plunger rod according to FIG. 6a is compared with a conventional plunger rod according to FIG. 8b. The weight of the inventive plunger rod is 16 g (FIG. 8a); the weight of the conventional plunger rod is 17 g (FIG. 8b). The torsional displacement of the inventive plunger rod is lower by a factor of 4 that that of the plunger rod according to the state of the art.

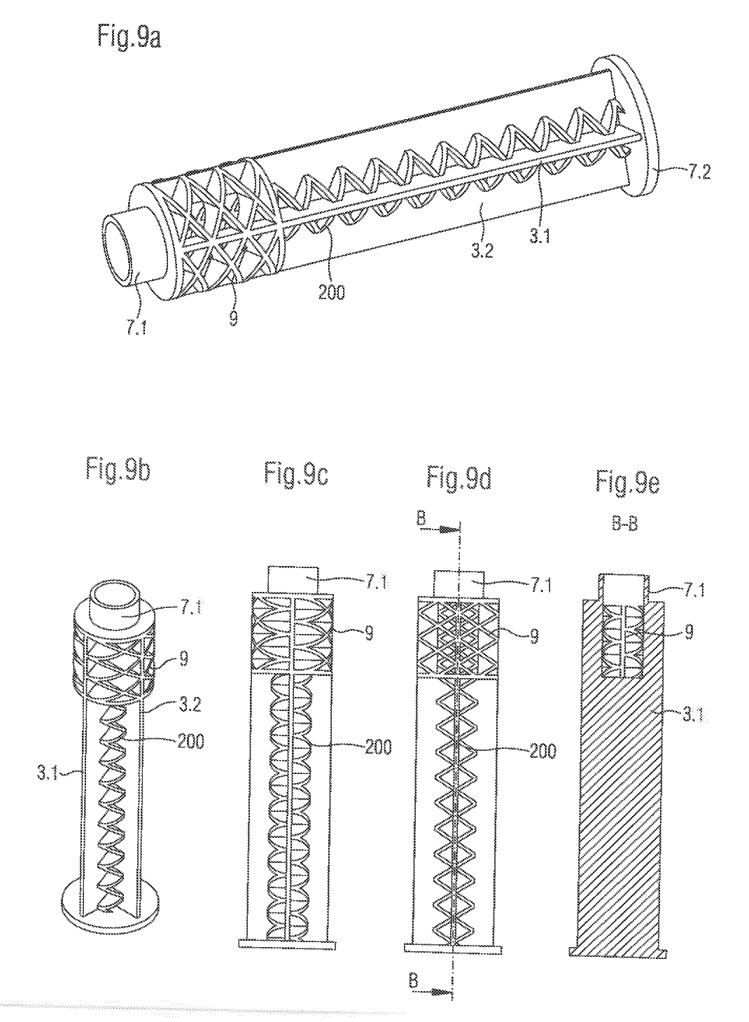

FIG. 9a illustrates another embodiment of a plunger rod according to the invention. The embodiment according to FIG. 9a does not include any tubular segments, but instead of the tubular segment 5 or segments 50.1, 50.2, 50.3, 50.4, 50.5, 50.6, 50.7 it has braces 200 that can be designed cross-shaped, like the reinforcement elements in the first section of the plunger rod. The reinforcement elements in the front plunger rod section can be designed diagonally to the torsional axis, as in FIG. 1a and serve to prevent canting of the syringe. Front plunger rod end 7.1 represents a segment with a thread. The thread in turn accommodates a plunger plug. The connection between plunger plug and plunger rod can be produced via an undercut. Rear plunger rod end 7.2 is designed as a thrust plate. Braces 200 in place of the tubular segment in the embodiment according to FIG. 9a provide the cross-sectional surface outside the torsional axis to increase the torsional or bending rigidity in the rear section of the plunger rod toward rear plunger rod end 7.2.

FIGS. 9b-9e illustrate detailed views of the plunger rod according to FIG. 9a. Same components as in FIG. 9a are identified with same reference numbers. FIG. 9e shows a sectional view along line B-B in FIG. 9d. FIG. 9d shows the arrangement of reinforcement ribs 200 for increasing the torsion and bending rigidity of the plunger rod.

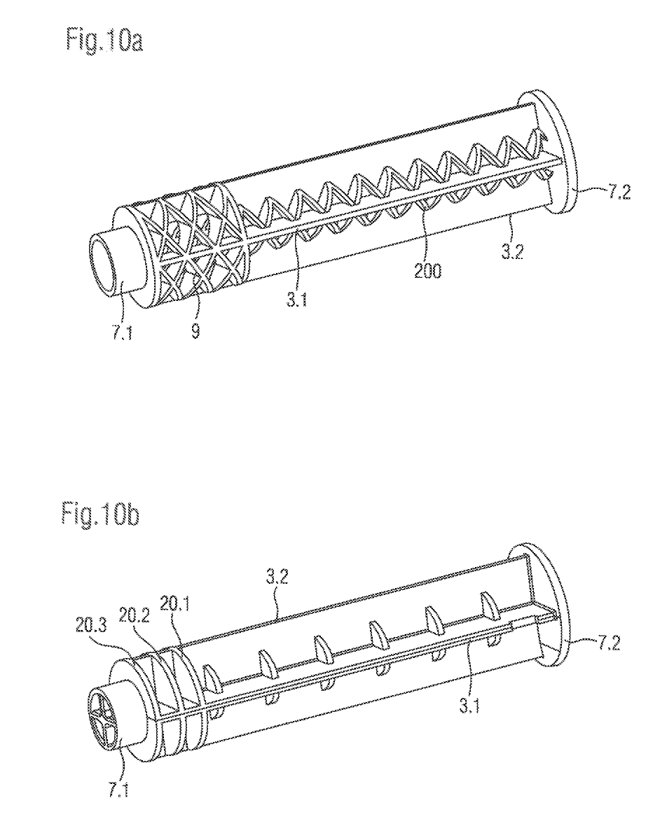

FIGS. 10a and 10b illustrate again a comparison of the inventive plunger rod according to FIG. 9b, with a plunger rod according to the state of the art in FIG. 10b. The plunger rod according to the state of the art has a weight of 17 g (FIG. 10b), the inventive plunger rod per FIG. 10a has a weight of 16.78 g. The torsional stress of the inventive plunger rod is less by a factor of 2 than that of the plunger rod according to the state of the art; the torsional displacement is lower by a factor of 4.

To keep the weight of the plunger rod low, the plunger rod may consist of plastic. The plunger rod consisting of plastic can be used in a plastic syringe as well as in a glass syringe.

Plastic materials such as thermoplastic materials may be used. One possible thermoplastic material can be polyethylene (PE) or polyoxymethylene (POM) or polystyrene (PS). Another polymer can be polypropylene (PP). Hard polymers, such as cycloolefin copolymer (COC) or cycloolefin polymer (COP) are used in particular as materials for the syringe barrel. To be able to use the syringe barrel for storage of pharmaceutical products, the materials of the syringe barrel can be sterilized at temperatures >100° C., for example 121° C., for example at a maximum of 180° C. Whereas cycloolefin polymer (COP) and cycloolefin copolymer (COC) cycloolefin copolymer (COC) cycloolefin copolymer (COC) are hard materials in which pharmaceutical media can be stored for several months, polypropylene or polyethylene are a softer material which can absorb stresses but which, due to its diffusion characteristics may not permit storage of pharmaceutical products over a longer period of time if used as the material for the syringe barrel.

If the plunger rod is designed as a hollow barrel in a thermoplastic material, the plunger rod thus produced in an injection molding process can be produced—with the assistance of an injection mold—with a long core that for example is inserted into the plunger rod from the plunger plug side. Alternatively, a closed hollow barrel could also be produced from a plastic with the assistance of an internal gas pressure process as a special injection molding process.

By using a plastic material instead of a glass material, as disclosed in U.S. Pat. No. 2,392,104 B, considerable weight reductions can be achieved, for example if the plunger rod is a plastic component.

The current invention cites for the first time designs of plunger rods that have higher torsional and bending rigidity compared to conventional plunger rods, as known from the state of the art, for example from U.S. Pat. No. 7,824,380. Moreover, tipping of the plunger rod inside the syringe barrel is countered.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A plunger rod for medical packaging such as syringe barrels or cartridges, said plunger rod comprising:
a first and a second plunger rod end;
a torsional axis; and
at least one component, each said component including at least one cross sectional surface which is located at a distance from the torsional axis and increases a bending- and torsional rigidity of the plunger rod.

2. The plunger rod according to claim 1, wherein the plunger rod is made from a plastic material.

3. The plunger rod according to claim 1, wherein the plunger rod is made from a thermoplastic material including at least one of a polypropylene (PP), a polystyrene (PS), a polyethylene (PE), and a polyoxymethylene (POM) material.

4. The plunger rod according to claim 1, wherein the plunger rod further includes a plurality of reinforcement elements, at least in sections, arranged diagonally opposite the torsional axis.

5. The plunger rod according to claim 4, wherein said plurality of reinforcement elements are in the form of a plurality of reinforcement ribs that increase the bending-and torsional rigidity of the plunger rod.

6. The plunger rod according to claim 5, wherein said plurality of reinforcement ribs each are tilted relative to the torsional axis at an angle α that is tilted >10°.

7. The plunger rod according to claim 1, wherein said plunger rod further includes a cross-shaped cross section.

8. The plunger rod according to claim 1, wherein said plunger rod further includes at least one tubular segment or partial section, wherein said at least one tubular segment or partial section is a hollow tubular segment or partial section.

9. The plunger rod according to claim 8, wherein said plunger rod further includes a plurality of reinforcement ribs, at least in sections, arranged diagonally opposite the torsional axis.

10. The plunger rod according to claim 9, wherein said plunger rod further includes several tubular segments or partial segments.

11. The plunger rod according to claim 8, wherein said at least one tubular segment or partial section has a tube diameter, wherein the tube diameter is approximately consistent with an outside diameter of the plunger rod.

12. The plunger rod according to claim 8, wherein said at least one tubular segment or partial section has a tube diameter, wherein the tube diameter is 10 to 90% of an outside diameter of the plunger rod.

13. The plunger rod according to claim 8, wherein a plurality of additional reinforcement elements are arranged at least one of outside and inside of the at least one tubular segment, wherein the plurality of additional reinforcement elements each are at least one of cross-shaped and honeycomb shaped.

14. The plunger rod according to claim 1, wherein the first plunger rod end includes a plunger plug and at least one support element.

15. The plunger rod according to claim 1, wherein the second plunger rod end includes a pressure plate.

16. A syringe barrel with a syringe cone, comprising:
a plunger rod that is guided in said syringe barrel, said plunger rod includes:
a first and a second plunger rod end;
a torsional axis; and
at least one component, each said component including at least one cross sectional surface which is located at a distance from the torsional axis and increases a bending- and torsional rigidity of the plunger rod.

17. A cartridge, comprising:
a plunger rod that is guided in said cartridge, said plunger rod includes:
a first and a second plunger rod end;
a torsional axis; and
at least one component, each said component including at least one cross sectional surface which is located at a distance from the torsional axis and increases a bending- and torsional rigidity of the plunger rod.
